(19) United States
(12) Patent Application Publication
(10) Pub. No.: US 2008/0306311 A1
(43) Pub. Date: Dec. 11, 2008

(54) Method for Producing an Alpha-Chiral Chloromethyl Compound in a Pure Form

(75) Inventors: Juergen Daeuwel, Heidelberg (DE); Volker Ruediger Sporys, Leimen (DE); Martin Voelkert, Ludwigshafen (DE); Holger Buehler, Martigny (CH)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/158,543

(22) PCT Filed: Dec. 13, 2006
(86) PCT No.: PCT/EP2006/069632

§ 371 Date: Jun. 20, 2008

(30) Foreign Application Priority Data
Dec. 23, 2005 (EP) 05112933.6

(51) Int. Cl. C07C 33/46

(52) U.S. Cl. 568/812

(57) ABSTRACT

The present invention relates to a process for preparing a specific α-chiral chloromethyl compound in pure or enriched form by distillative removal of the compound mentioned from substance mixtures which comprise this compound and higher-boiling impurities. The α-chiral chloromethyl compound in question is present in crystalline form at room temperature and is a central intermediate for the preparation of a class of medicaments.

### TECHNICAL FIELD OF THE INVENTION

The present invention relates to a process for preparing a specific α-chiral chloromethyl compound in pure or enriched form by distillative removal of the compound mentioned from substance mixtures which comprise this compound and higher-boiling impurities. The α-chiral chloromethyl compound in question is present in crystalline form at room temperature and is a central intermediate for the preparation of a class of medicaments.

EP 0 678 503 described 8-amino-γ-hydroxy-ω-arylalkenecarboxamides which have renin-inhibiting properties and can be used as an antihypertensive in pharmaceutical formulations.

WO 01/09083 describes a process for preparing the 8-amino-γ-hydroxy-ω-arylalkenecarboxamides mentioned. The central intermediate mentioned is the compound of the formula (Ia)

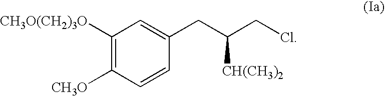

This is obtained by chlorinating the corresponding alcohol of the formula (IIa)

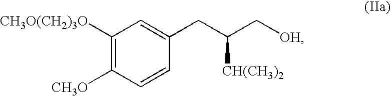

the chlorinating reagents used being carbon tetrachloride and trioctylphosphine. After extracted workup, the resulting reaction product is purified first by flash chromatography and then by crystallization from hexane at −50° C.

In Tetrahedron Letters 41 (2000) 10085-10089, H. Rüeger et al. describe the preparation of the compound of the formula (Ia) by reacting the compound of the formula (IIa) with thionyl chloride in the presence of pyridine and chloroform as a solvent with a yield of 70%.

In Tetrahedron Letters 41 (2000) 10091-10094, D. A. Sandham et al. describe the preparation of the compound of the formula (Ia) by reacting the compound of the formula (IIa) with phosphoryl chloride in the presence of DMF and toluene as solvents with a yield of 78%.

Against the background of the requirements made on a process, performable in an economically viable manner on the industrial scale, for preparing a medicament intermediate, the process described cannot be considered to be satisfactory. Especially the use of toxic reagents and the complicated multistage purification of the crude product are problematic in this context.

### OBJECT OF THE INVENTION

It was thus an object of the present invention to provide a process for preparing the compound of the formula (I) in pure or enriched form, which does not have the aforementioned disadvantages and which makes it possible to obtain the compound of the formula (I) in high yield and purity.

### DESCRIPTION OF THE INVENTION AND OF THE PREFERRED EMBODIMENTS

The object is achieved in accordance with the invention by the provision of a process for preparing optically active compounds of the formula (I)

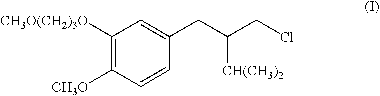

in pure or enriched form by distillatively removing the compound of the formula (I) from substance mixtures comprising the optically active compound of the formula (I) and higher-boiling impurities.

The process according to the invention is especially suitable for preparing the compound of the formula (Ia)

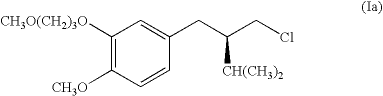

in pure or enriched form by distillatively removing the compound of the formula (Ia) from substance mixtures comprising the optically active compound of the formula (Ia) and higher-boiling impurities.

Suitable substance mixtures to be used in accordance with the invention are in particular those which consist to an extent of from about 25 to about 99% by weight, preferably to an extent of from about 50 to about 98% by weight, more preferably to an extent of from about 75 to about 97% by weight, even more preferably to an extent of from about 85 to about 97% by weight and most preferably to an extent of from about 90 to about 97% by weight, of the compound of the formula (I) or of the compound of the formula (Ia).

In addition to the compound of the formula (I) or (Ia) to be prepared in pure or enriched form, the substance mixtures mentioned also comprise higher-boiling impurities and, if appropriate, also lower-boiling impurities, for example solvent residues or low molecular weight by-products of the preceding synthesis stages.

The process according to the invention serves to prepare optically active compounds of the formula (I) or (Ia) in pure or enriched form. The expression “in pure or enriched form” means either that the compound of the formula (I) or (Ia) is obtained in pure form or is obtained in the form of a substance mixture which has a higher content of a particular compound of the formula (I) or (Ia) than the substance mixtures used in accordance with the invention.

The compound of the formula (I) or (Ia) in pure form is understood to mean the particular compound having a purity of at least about 98% by weight, preferably from about 99.5 to about 99.9% by weight. In the context of the present invention, the compound of the formula (I) or (Ia) in enriched form is preferably understood to mean substance mixtures which consists to an extent of from about 90 to about 99.9% by weight, preferably from about 95 to about 99.9% by weight, more preferably from about 95 to about 99.9% by weight and most preferably to an extent of from about 97 to about 99.9% by weight, of the compound of the formula (I) or (Ia).

The pure or enriched compounds of the formula (I) or (Ia) are obtained in optically active form. The enantiomeric excess of the particular compound of the formula (I) or (Ia) obtained preferably corresponds substantially to that of the compound of the formula (I) or (Ia) present in the substance mixture used in accordance with the invention. The compound of the formula (I) or (Ia) is preferably obtained in pure or enriched form with an enantiomeric excess which is at least 85%, more preferably at least 90%, most preferably at least 95% of the enantiomeric excess of the compound of the formula (I) or (Ia) used.

The process according to the invention is preferably carried out in such a way that the distillative removal is carried out at a pressure in the range from about 0.0001 mbar to about 10 mbar, preferably from about 0.001 to about 5 mbar and more preferably from about 0.001 to about 0.1 mbar.

Depending on the pressure selected, the inventive distillative removal can be carried out at temperatures in the range from about 50° C. to about 250° C., preferably from about 80 to about 220° C.

The inventive removal of the compound of the formula (I) or (Ia) can be carried out in a multitude of configurations known to those skilled in the art, as described in detail, for example, in handbooks of chemical technology such as Ullmann or Winnacker-Küchler. Preference is given to carrying out the inventive removal in the form of a continuous distillation.

Particularly preferred embodiments of the process according to the invention are so-called short-path or molecular distillations in which very short and straight-line paths between evaporator and condenser surfaces are employed. Suitable evaporators are in particular short-path evaporators, thin-film evaporators or falling-stream evaporators. Particularly suitable apparatus also includes commercially available molecular distillation apparatus as sold by specialist suppliers. Alternatively, rectification in the moderate vacuum range is also possible.

It is also possible to operate a plurality of the apparatus mentioned in series, for example in order to first remove any volatile impurities or lower-boiling impurities still present. It has been found to be advantageous to undertake a degassing of the substance mixture used before the distillative removal of the desired compound.

The process according to the invention allows the preparation of the compound of the formula (I) or (Ia) in pure or enriched form. The present invention accordingly also relates to a process for purifying the compound of the formula (I) or (Ia) by distillatively removing the compound of the formula (I) from substance mixtures comprising the optically active compound of the formula (I) and higher-boiling impurities.

In addition to the higher-boiling impurities mentioned, the substance mixtures to be used in accordance with the invention may also comprise lower-boiling compounds, i.e. compounds which have a lower boiling point than the compound of the formula (I) or (Ia). These lower-boiling compounds, for example solvent residues, excess reagents or low molecular weight by-products of the preceding synthesis stages, may be obtained as first fractions in the course of the inventive distillative removal and thus likewise be removed from the compound of the formula (I) or (Ia).

In the context of the present invention, the term higher-boiling impurities is understood to mean those compounds which have a higher boiling point than the compound of the formula (I) or (Ia). The higher-boiling compounds mentioned may also be by-products of the synthesis sequence for preparing the compound of the formula (I). For example, the substance mixtures to be used in accordance with the invention may comprise dimerization products of the compound of the formula (I), for example the compound of the formula (III)

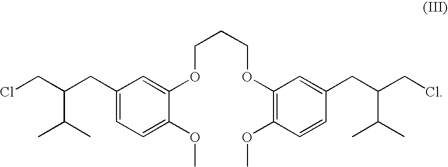

Further possible higher-boiling dimerization products are, for example, those in which the two halves of the molecule are joined to one another via the aromatics by a disulfide bridge.

Such higher-boiling impurities are formed to a small degree, for example, in the preparation of the compound of the formula (I) in optically active form by reaction of an optically active alcohol of the formula (II)

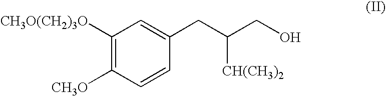

with thionyl chloride and N,N-dimethylformamide.

In a preferred embodiment, the present invention therefore relates to the process described above, wherein substance mixtures obtainable by reacting an optically active alcohol of the formula (II)

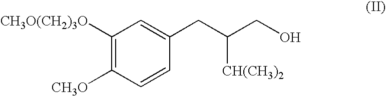

with thionyl chloride and N,N-dimethylformamide are used. Accordingly, the present invention relates to a process for preparing optically active compounds of the formula (I)

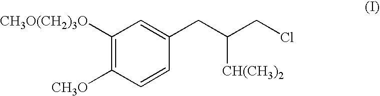

in pure or enriched form, comprising the steps of
a) reacting an optically active alcohol of the formula (II)

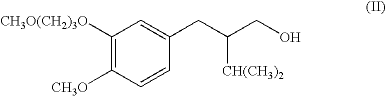

or (IIa) with thionyl chloride and N,N-dimethylformamide to obtain the compound of the formula (I) or (Ia) and
b) distillatively removing the compound of the formula (I) from the substance mixtures which are obtained in step a) and comprise the optically active compound of the formula (I) and higher-boiling impurities.

The present invention also relates to a process for preparing the compound of the formula (I) or (Ia) by reacting the compound of the formula (II) or (IIa) with thionyl chloride and N,N-dimethylformamide.

In the context of the process according to the invention for preparing the compound of the formula (I) in optically active form by reacting the compound of the formula (II) in optically active form, the optically active compound of the formula (II) and thionyl chloride are reacted, preferably in a molar ratio in the range from about 1:1 to about 1-5, more preferably from about 1:1.1 to about 1:2.

The reaction is carried out in the presence of N,N-dimethylformamide, in which case N,N-dimethylformamide and thionyl chloride are used preferably in a molar ratio in the range from about 0.01:1 to about 1:1, more preferably in the range from about 0.03:1 to 0.1:1.

The reaction is preferably carried out in such a way that a solution of the optically active alcohol of the formula (II) is initially charged in a suitable solvent inert under the reaction conditions, for example benzene, toluene, xylenes, ethers, for example diethyl ether, THF, dioxane and the like, halogenated solvents, for example methylene chloride, chloroform, 1,2-dichloroethane and the like, preferably with toluene, together with the selected amount of N,N-dimethylformamide, and the selected amount of thionyl chloride is added at a temperature of from about 80 to about 100° C. The reaction is typically substantially complete after about from one to about 5 h, often after about 2 h. After the reaction has ended, excess thionyl chloride is removed, advantageously by distillation under reduced pressure, and the residue is neutralized by adding a suitable base, for example aqueous sodium hydroxide solution or aqueous NaHCO₃ solution. The further workup of the resulting reaction mixture can be undertaken by methods known to those skilled in the art.

The process according to the invention for preparing the compound of the formula (I) or (Ia) in pure or enriched form opens up an unexpectedly efficient route to the compound mentioned, specifically to the compound of the formula (Ia) in a form which takes account of the requirements which are made on a medicament intermediate. It is distinctly superior to the known processes for purifying the compound mentioned by crystallization, especially with regard to the number of process steps and the yield and purity of the product, since especially higher-boiling, structurally similar impurities with high tendency to crystallize can only be removed insufficiently from the desired product by conventional crystallization.

The examples which follow serve to illustrate the invention without restricting it in any form:

### EXAMPLE 1

A 1 m³ enameled steel tank was charged with 440 kg of an about 29% solution of the alcohol of the formula (II) with an enantiomeric excess of 99.2% ee in toluene and 3.5 kg of N,N-dimethylformamide (DMF). At 90° C., 77 kg of thionyl chloride were metered in, and the tank contents were stirred for a further 2 hours and cooled to 40° C. The excess thionyl chloride was distilled off under reduced pressure. At from 2 to 10° C., the distillation bottoms were added, in a 1 m³ HC tank, to 100 l of 15% sodium hydroxide solution, the aqueous lower phase was removed and the organic upper phase was washed with 100 l of demineralized water. 124 kg of the compound of the formula (Ia) were obtained as an about 30% solution in toluene. The enantiomeric excess of the resulting product was 98.5% ee.

### EXAMPLE 2

In a 4 m³ enameled steel tank, the solvent was distilled off under reduced pressure from 1300 kg of an about 30% solution of the compound of the formula (Ia) in toluene. The remaining bottoms were admixed at 50° C. with 1600 kg of methanol, cooled to 20° C., seeded with crystals and cooled further to −10° C. at a rate of 10 K/h. The resulting crystal slurry was removed at −10° C. through a process filter, washed three times with 200 kg of cooled methanol and dried under reduced pressure. 250 kg of the compound of the formula (Ia) were obtained in crystalline form, corresponding to a yield of 64%. The enantiomeric excess was over 99.8% ee. The purity of the reaction product was analyzed by means of HPLC by the following method:

### Column: Waters Symmetry C18 5 μm, 150×3 mm

Eluent: A) 0.2% by vol. of H₃PO₄ in water; B) CH₃CN
Gradient (based on eluent B): 0 min (45%) 12 min (95%) 13 min (95%) 13.1 min (45%)
Flow rate: 1.2 ml/min, temperature: 60° C., injection volume: 5 μl
Detection: UV detector at 230 nm

According to HPLC analysis, the material comprised 98.1 area % of the compound (Ia) and 0.67 area % of a higher-boiling secondary component. In this method compound (Ia) eluted at 6.61 min and the secondary component at 11.89 min.

### EXAMPLE 3

500 g of the crystallized material from example 2, comprising approx. 98.1% of the compound of the formula (Ia), were distilled at 0.1 mbar and evaporator temperature 180° C. using a KDL 5 short-path evaporator from UIC GmbH, Alzenau-Hörstein. 430 g (87% yield) of bright yellow distillate having a content of over 99% and an enantiomeric excess of 99.8% ee were obtained.

### EXAMPLE 4

500 g of the crystallized material from example 2, comprising approx. 98.1% of the compound of the formula (Ia), were distilled at 0.001 mbar and evaporator temperature 100° C. using a short-path evaporator from UIC GmbH. 456 g (92% yield) of bright yellow distillate having a content of over 99% and an enantiomeric excess of 99.8% ee were obtained.

### EXAMPLE 5

500 kg of the crystallized material from example 2, comprising approx. 98.1% of the compound of the formula (Ia) and 0.67 area % of a secondary component, were distilled continuously in three stages (degassing at 1 mbar, 120° C., thin-film evaporator 0.1 mbar, 120° C., short-path evaporator, 0.01 mbar, 140° C.) to remove low- and high-boiling secondary components. The low boilers were removed in the course of the degassing and thin-film evaporation, the high boilers in the subsequent short-path distillation. 420 kg (85% yield) of bright yellow distillate having a content of 99.4 area % according to the HPLC method specified in example 2 were obtained, in which the secondary component was no longer detectable. The enantiomeric excess was over 99.8% ee.

1-15. (canceled)

16. A process for preparing an optically active compound of formula (I) 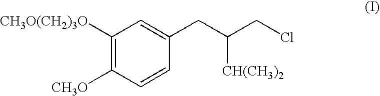
in pure or enriched form comprising distillatively removing said optically active compound of formula (I) from substance mixtures comprising said optically active compound of the formula (I) and higher-boiling impurities.

17. The process of claim 16, wherein said optically active compound of formula (I) is a compound of formula (Ia)

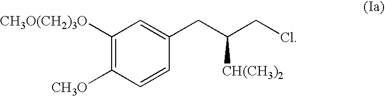

18. The process of claim 16, wherein said distillative removal is carried out at a pressure in the range of from 0.0001 mbar to 10 mbar.

19. The process of claim 16, wherein said distillative removal is carried out at a temperature in the range of from 50° C. to 250° C.

20. The process of claim 16, wherein said distillative removal is carried out in the form of a continuous distillation.

21. The process of claim 20, wherein said continuous distillation is carried out in the form of a molecular distillation or by means of a short-path evaporator, thin-film evaporator, or a falling-stream evaporator or by a rectification in the moderate vacuum range.

22. The process of claim 16, wherein said substance mixtures further comprise lower-boiling impurities.

23. The process of claim 16, wherein said substance mixtures comprise from 25% to 99% by weight of said optically active compound of formula (I).

24. The process of claim 16, wherein the optically active compound of formula (I) obtained has a purity of from 95% to 99.9% by weight.

25. The process of claim 16, wherein the optically active compound of formula (I) obtained has a purity of from 98% to 99.9% by weight.

26. The process of claim 16, wherein said higher-boiling impurities are by-products of the synthesis of said optically active compound of formula (I).

27. The process of claim 16, wherein said substance mixtures are obtained by reacting an optically active alcohol of formula (II) 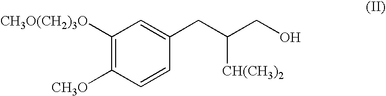
with thionyl chloride and N,N-dimethylformamide.

28. A process for preparing an optically active compound of formula (I) 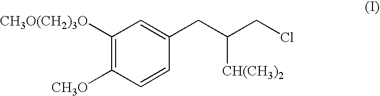
comprising reacting an optically active alcohol of formula (II) 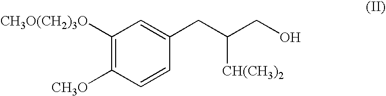
with thionyl chloride and N,N-dimethylformamide.

29. The process of claim 28, wherein said compound of the formula (II) and thionyl chloride are used in a molar ratio in the range of from 1:1 to 1:5.

30. The process of claim 28, wherein said thionyl chloride and N,N-dimethylformamide are used in a molar ratio in the range of from 1:0.01 to 1:1.
